(19) United States
(12) Patent Application Publication
(10) Pub. No.: US 2010/0075969 A1
(43) Pub. Date: Mar. 25, 2010

(54) Angiogenesis

(75) Inventor: Christine Elaine Pullar, Leicester (GB)

Correspondence Address: Fox Rothschild LLP;Blue Bell, 2000 Market Street, Tenth Floor, Philadelphia, PA, 19103, US

(73) Assignee: UNIVERSITY OF LEICESTER, Leicestershire (UK)

(21) Appl. No.: 12/519,937

(22) PCT Filed: Dec. 19, 2007
(86) PCT No.: PCT/GB2007/050770

§ 371 Date: Jun. 18, 2009

(30) Foreign Application Priority Data
Dec. 19, 2006 (GB) 0625270.4

(51) Int. Cl. A61K 31/137; A61K 31/138; A61K 31/5377; A61P 35/00

(52) U.S. Cl. 514/236.2; 514/653; 514/651

(57) ABSTRACT

The invention provides medicaments comprising an agent, which selectively modulates β2-adrenergic receptor conformation, or receptor activity, or activation thereof. The medicaments may be used for treating diseases that are characterised by disorganized and/or abnormal vasculature, such as cancer and metastatic disease. The medicaments may also be used for promoting organized/normal branched vasculature.

The present invention relates to angiogenesis, and in particular to medicaments which may be used for treating diseases that are characterised by disorganized/abnormal (ie unbranched) vasculature, such as cancer and metastatic disease. The invention also provides methods of treating subjects suffering from such diseases. The invention further provides medicaments and methods for promoting organized/normal (ie branched) vasculature, which have far-reaching medical applications.

Angiogenesis refers to the growth of new blood vessels, and is essential for the physiological process of wound healing. However, angiogenesis may be detrimental to the health of a subject because it can support neoplastic conditions, such as cancer, and also inflammatory conditions. Tumour vasculature is known to be characterised by an increased permeability, disorganized vascular networks, abnormal cell morphology and also variable vessel density. The disorganized tumour vasculature results in an inadequate blood supply to the tumour causing tumour hypoxia. Hypoxic tumours are highly resistant to chemotherapy and radiation therapy, and correlate with poor patient prognosis.

Therapies targeted towards pathologic or inappropriate angiogenesis are believed to be possible. However, ongoing clinical trials have proved to be disappointing due to the lack of information in relation to the underlying specific anti-angiogenic molecular mechanisms. Another problem with current clinical trials is due to poor drug delivery to the tumour, which is thought to be due to the immature and disorganized vasculature in the tumour.

One of the best ways to prevent metastatic disease is the early detection of a tumour. Transepithelial potentials and electric fields are critical guidance cues in development and regeneration, and their loss in cancerous tissue may play a role in metastasis. One of the current promising diagnostic tests for breast cancer capitalises on the altered electrical potential of the rapidly proliferating cancerous tissue. Regions of epithelial electrical depolarisation occur within the breast parenchyma as the rapidly proliferating cells become depolarised in comparison with normal cells. Indeed, polarised epithelial cells lose their transepithelial electric potential during carcinogenesis. These areas of depolarisation can be detected using specially designed sensors placed on the skin and, it is hoped, could alleviate unnecessary testing and anxiety for the patients.

More recently, “stress” has been shown to promote chemoresistance in breast cancer, invasion of ovarian cancer cells, promotion of tumour growth and also angiogenesis in a mouse model of ovarian carcinoma. Research has suggested that stress-induced tumour growth and metastasis may be mediated by the β2-adrenergic receptor, and could be blocked by a non-selective β-blocker. Non-selective β-blockers are molecules which block all three of the β-adrenergic receptors (including the β1-adrenergic receptor, β2-adrenergic receptor and β3-adrenergic receptor), and are used to treat various cardiovascular conditions, such as hypertension, angina and cardiac arrhythmia. The stress-induced ligands of the β2-adrenergic receptor, adrenaline and noradrenaline, can also drive the metastatic development of prostate cancer cells in nude mice, and receptor blockade, by a non-selective β-blocker, inhibits the process. This work highlights the potential of using non-selective β-blockers as a possible treatment for prevention of stress-induced tumour growth and metastasis.

However, problems associated with this work are that the researchers used non-selective β-blockers, such that all three of the β-adrenergic receptors (β1-adrenergic receptor, the β2-adrenergic receptor and also the β3-adrenergic receptor) were blocked. This can be particularly problematic because blockade of β1, β2- and β3-adrenergic receptors significantly alters cardiac function and lipid metabolism, and this is detrimental to a patient. Additionally, the researchers failed to observe any changes to tumour vasculature in the presence of a non-selective β-blocker, and assumed that the ligand had merely blocked the receptor from the stress-mediated induction of tumour growth.

It is therefore an object of the present invention to obviate or mitigate one or more of the problems of the prior art, whether identified herein or elsewhere, and to provide improved medicaments, which may be used in methods for treating disease conditions characterized by disorganized or unbranched vasculature. Furthermore, due to the number of medical contexts in which disorganized blood vessel formation or vascularisation may be considered undesirable, there remains a need for new medicaments that are capable of promoting organized or branched blood vessel formation.

The inventor of the present invention believes that one barrier to drug efficacy used for treating tumours appears to be the associated disorganized tumour vasculature, which hinders the delivery of a drug to the tumour, resulting in low tumour-drug concentrations and hence, low systemic toxicity. The inventor therefore set out to address the two major problems inherent with currently available approaches to cancer treatment, ie the lack of information on the underlying anti-angiogenic molecular mechanisms, and poor drug delivery to tumours due to the disorganized and immature vasculature exhibited in tumours. As a model, the inventor focused her research on the β2-adrenergic receptor. However, in contrast to previous workers who investigated the efficacy of non-selective β-blockers, the inventor examined the efficacy of highly selective β2-adrenergic receptor modulators (ie agonists and antagonists), and monitored their effect on angiogenesis.

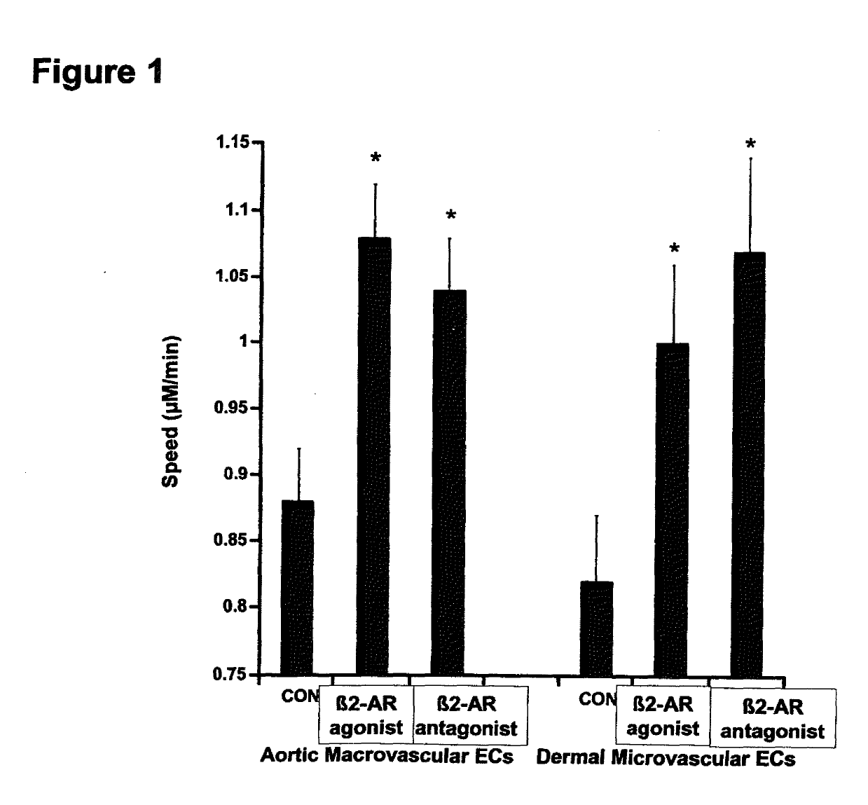

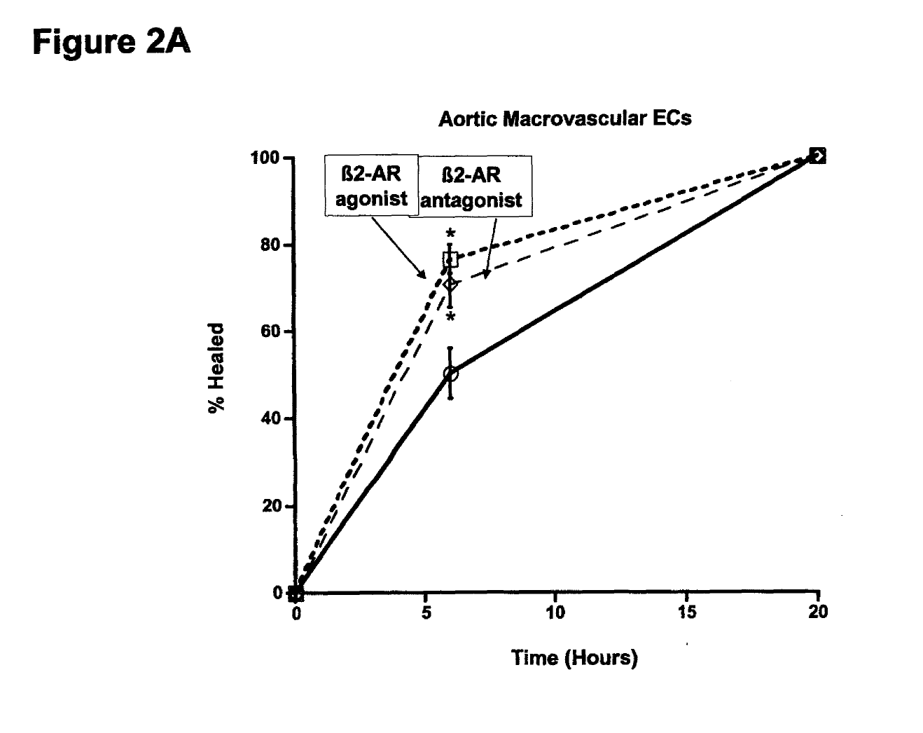

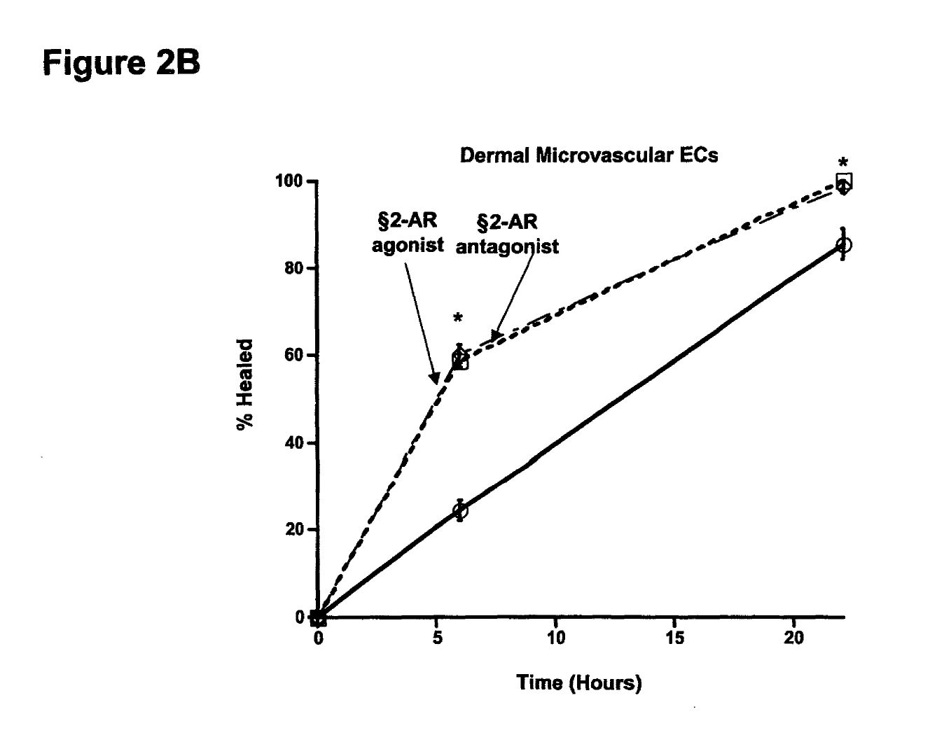

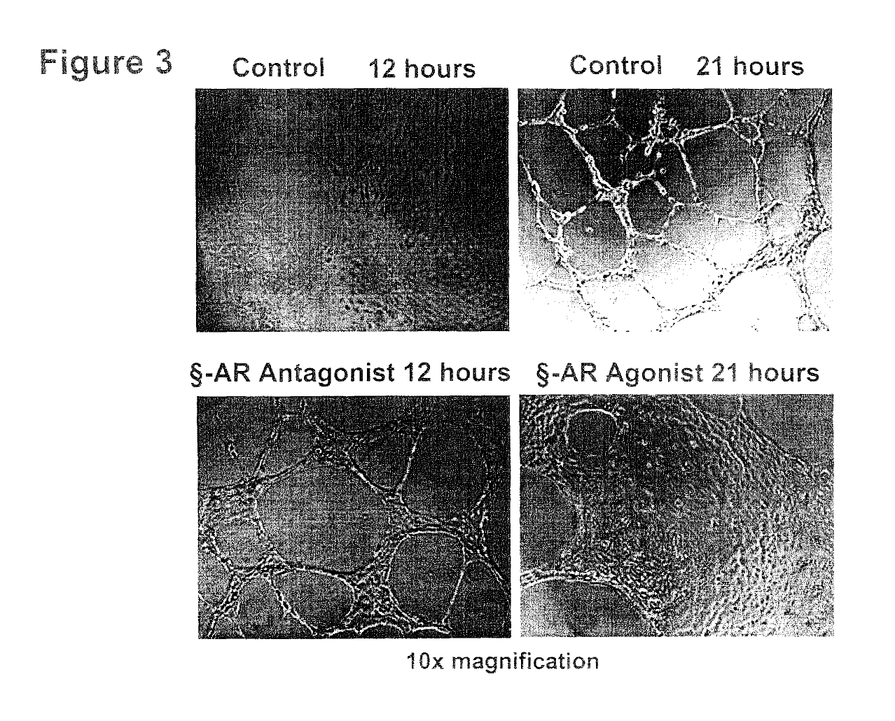

As described in the Examples, and as shown in FIGS. 1 and 2, the inventor has demonstrated that selective β2-adrenergic receptor agonists and antagonists increase the migration of endothelial cells in vitro. Furthermore, administration of the antagonist enhances the formation of tubule-like structures, as shown in FIG. 3, and administration of the agonist inhibits the formation of tubules. As shown in FIGS. 4a and 4b, the data show that organized vessel growth and angiogenesis, and vessel branching was promoted by the use of selective β2-adrenergic receptor antagonists. In contrast, disorganized tubule growth and a reduction in vessel branching was initiated by β2-AR agonists.

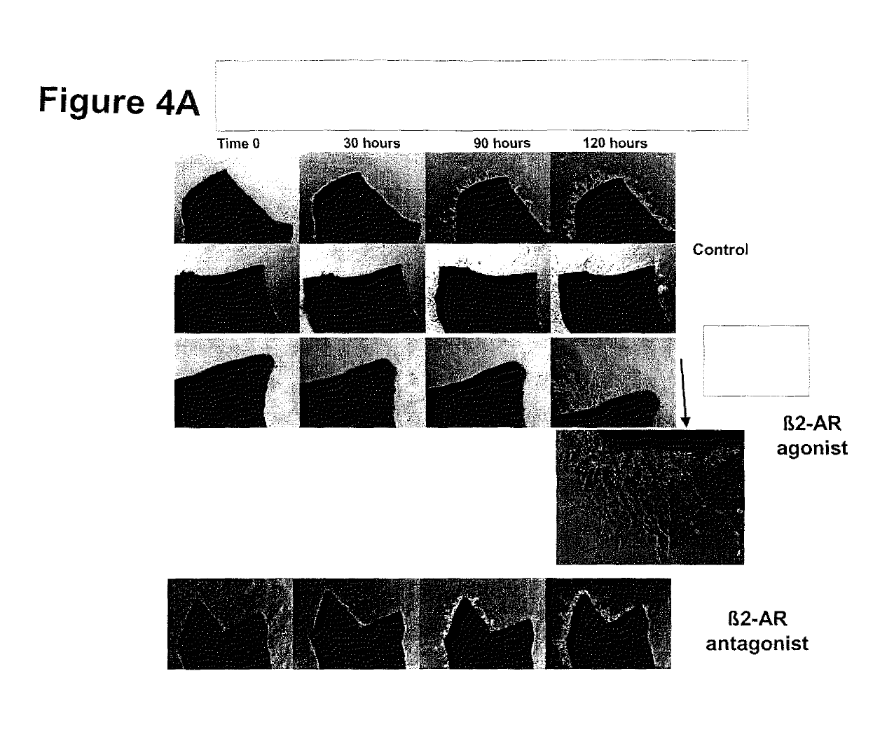

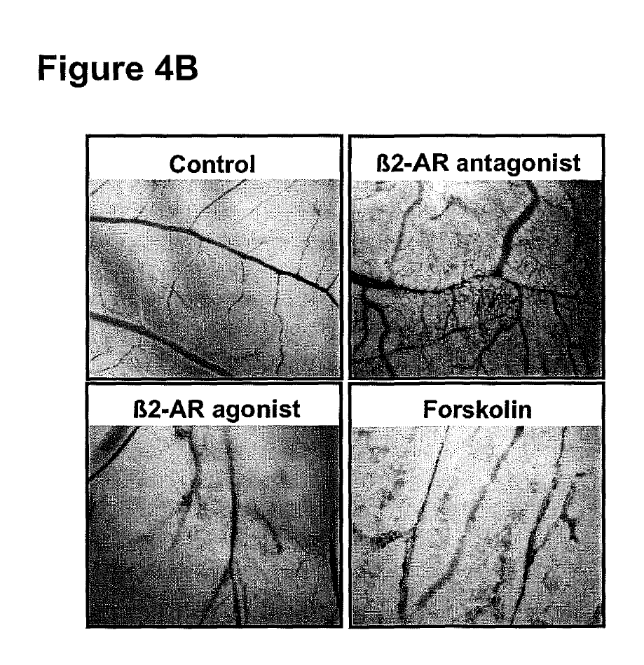

Additionally, the inventor has realized that tumours exhibit enhanced electric fields surrounding them, and FIG. 5 shows that endothelial cells are not influenced when exposed to an electric field, ie decreases the directional migration of the cells, and FIG. 6 shows that a β2-adrenergic receptor agonist also blinds a breast cancer cell line to an applied electric field. The inventor hypothesizes that this decrease in directional migration caused by the selective β2-adrenergic receptor agonist causes disorganized or unbranched vasculature, thereby promoting metastasis. Accordingly, while the inventor does not wish to be bound by any hypothesis, she believes that a specific β2-adrenergic receptor agonist promotes endothelial cell migration, while inhibiting vessel branching, thereby forming a disorganized/immature vasculature, which is characteristic of tumour vasculature, and that endothelial/cancer cells are not influenced by an endogenous local electric field set up across the cell membrane, thereby promoting metastasis.

As shown in FIGS. 1 and 2, the data also surprisingly demonstrates that the use of a highly selective β2-adrenergic receptor antagonist, such as ICI 118,551, results in the promotion of dermal microvascular and aortic macrovascular endothelial cell migration, and an organized, branched vasculature in a tumour or normal tissue. In particular, FIG. 3 shows that organized vasculature is formed only 12 hours after addition of the β2-adrenergic receptor-selective antagonist, which the inventor found to be most surprising. In addition, FIG. 4a shows that the antagonist causes the formation of shorter capillaries (when compared to those formed under the influence of the agonist), and that these capillaries form a more ordered vasculature. It is this organized angiogenesis, which the inventor believes allows the use of a β2-adrenergic receptor-selective antagonist in the treatment of a wide variety of disease conditions that are characterised by disorganized or unbranched vasculature. Finally, FIG. 4b demonstrates that the β2-AR antagonists promote angiogenesis, and vascular branching in vivo.

The data therefore clearly demonstrate that β2-adrenergic receptor-selective antagonists promote the formation of organized and branched angiogenesis. Hence, the inventor believes that a β2-adrenergic receptor-selective antagonist will be useful in clinical applications where it is beneficial to promote an ordered/branched vasculature or angiogenesis. The data also show that β2-adrenergic receptor-selective agonists increase the length of tubules, but inhibit branching. Hence, the inventor believes that using agonists will also have therapeutic applications.

The inventor believes that agents, which selectively modulate β2-adrenergic receptor conformation, or the activity of the receptor, or the activation of the receptor (ie alter the downstream signalling pathway controlled by the receptor, such as the cAMP cascade), may be used to promote organized vasculature. The inventor also believes that agents, which selectively modulate β2-adrenergic receptor conformation or activity or activation thereof may be used to treat diseases which are characterized by disorganized vasculature.

Therefore, in a first aspect, there is provided an agent, which selectively modulates β2-adrenergic receptor conformation, or receptor activity, or activation thereof, for use in the promotion of organized vasculature or for the treatment, amelioration or prevention of a condition characterized by disorganized vasculature.

In a second aspect, there is provided use of an agent, which selectively modulates β2-adrenergic receptor conformation, or receptor activity, or activation thereof, in the manufacture of a medicament for the promotion of organized vasculature, or for the treatment, amelioration or prevention of a condition characterized by disorganized vasculature.

In a third aspect, there is provided a method for promoting organized vasculature, or for treating, ameliorating or preventing a condition characterized by disorganized vasculature in a subject, the method comprising administering, to a subject in need of such treatment, a therapeutically effective amount of an agent, which selectively modulates β2-adrenergic receptor conformation, or receptor activity, or activation thereof.

The inventor believes that her findings have significant differences with the prior art because she used an agent, which selectively modulates the β2-adrenergic receptor, ie one which modulates the β2-adrenergic receptor only. This is in contrast to the use of a non-selective β2-adrenergic receptor inhibitor as disclosed in the prior art, such as a β-blocker, which blocks all three of the β1-, β2- and β3-adrenergic receptors simultaneously. Previous workers have failed to realize that both a β2-adrenergic receptor-selective agonist and a β2-adrenergic receptor-selective antagonist promote angiogenesis. However, the inventor of the present invention has now determined that a β2-adrenergic receptor-selective agonist stimulates an increase in blood vessel length and promotes a tortuous or disorganized vasculature lacking vessel branching, whereas a β2-adrenergic receptor antagonist promotes angiogenesis and vascular branching creating a more developed organized vasculature (as shown in FIG. 4b), and much quicker than in the absence of the antagonist, as shown in FIG. 3.

By the term “selectively modulates”, we mean that the agent alters receptor conformation, or blocks the β2-adrenergic receptor activity or activation thereof to a greater extent, or at lower doses, than other types of adrenergic receptors, ie α1-, α2-, β1-, or β3-adrenergic receptors. Hence, the agent is selective only for the β2-adrenergic receptor. As mentioned herein, a β-blocker inhibits the β1-, β2-, and β3-adrenergic receptors, and so does not selectively inhibit the β2-adrenergic receptor. Similarly, α-blockers inhibit both the α1- and α2-adrenergic receptors, and so do not selectively inhibit the β2-adrenergic receptor.

By the term “modulate β2-adrenergic receptor conformation”, we mean the agent is capable of altering the three-dimensional shape and configuration of the receptor between its active and inactive conformations.

The skilled technician will appreciate what is meant by the term “β2-adrenergic receptor”. These receptors are known in the art and have been reviewed in Johnson M, (J Allergy Clin. Immunol. 2006 117 18-24). However, for the avoidance of doubt, adrenergic receptors are a class of G protein-coupled receptors which bind and are activated by their endogenous ligands, the catecholamines, adrenaline and noradrenaline. The adrenergic receptors fall into 5 types: α1, α2, β1, β2, and β3, and the present invention is concerned with the β2-adrenergic receptor (ie β2-AR).

The DNA and protein sequences for the human β2-adrenergic receptor are available on freely accessible databases and are discussed in Kobilka et al (1987 PNAS, 84, 46-50). The chromosomal location for the gene encoding the β2-adrenergic receptor is chromosome Sq 31-32.

Several classes of agent may be used in accordance with the invention. For example, the agent which selectively modulates β2-adrenergic receptor conformation, or the activity of the receptor, or the activation of the receptor, may be a positive modulator, which may be capable of:
(i) altering the conformational state of the receptor, for example by stabilizing the active conformation of the receptor and/or maintaining the receptor in its active conformation to thereby allow the receptor to bind its natural ligand, ie the catecholamines.
(ii) binding to the β2-adrenergic receptor, and increasing, promoting or augmenting transmission at the receptor;
(iii) promoting or activating the downstream signalling pathways activated by the modulator binding to the receptor;
(iv) increasing, promoting or augmenting transcription, translation or expression of the β2-adrenergic receptor;
(v) increasing synthesis or release of the β2-adrenergic receptor, or agonists thereof, from intracellular stores; or
(vi) decreasing the rate of degradation of β2-adrenergic receptor, or agonists thereof.

Suitably, the binding affinity value (Ki value) of the positive modulator for the β2-adrenergic receptor is less than about 100 nM, more suitably less than 80 nM, and more suitably less than 50 nM. Preferably, the Ki value of the positive modulator for the β2-adrenergic receptor is less than 30 nM, more preferably less than 15 nM, and more preferably less than 10 nM.

A preferred positive modulator is a β2-adrenergic receptor-selective agonist.

By the term “agonist”, we mean a molecule that selectively binds to the β2-adrenergic receptor to initiate the signal transduction reaction.

Suitable β2-adrenergic receptor-selective agonists may include fenoterol, butoxamine, salbutamol, clenbuterol, formoterol, or salmeterol. However, a preferred β2-adrenergic receptor-selective agonist is salbutamol, as described in the Examples. Salbutamol is a highly selective β2-AR agonist, and will be known to the skilled technician.

The log K_{d} (dissociation constant) of salbutamol for β1 is −4.66, for β3 is −4.33, and for β2 is −6.12. Hence, the log K_{d} is much lower for β2-adrenergic receptor than for the β1- or the β3-adrenergic receptor. Salbutamol is therefore at least 29 times more selective for β2-AR than for the β1-adrenergic receptor, and 62 times more selective for β2-AR than for the β3-adrenergic receptor, and may therefore be described as being a β2-adrenergic receptor-selective agonist.

However, it is preferred that the agent, which selectively modulates β2-adrenergic receptor conformation, or the activity of the receptor, or the activation of the receptor, is a negative modulator, which may be capable of:
(i) altering the conformational state of the receptor, for example by destabilizing the active conformation of the receptor and/or maintaining the receptor in its inactive conformation to thereby prevent the receptor from binding its natural ligand, ie the catecholamines.
(ii) binding to the β2-adrenergic receptor, and preventing, decreasing or attenuating transmission at the receptor;
(iii) down-regulating or de-activating the downstream signalling pathways activated by the modulator binding to the receptor, for example by inhibiting or blocking the cAMP signalling cascade;
(iv) decreasing, preventing or attenuating transcription, translation or expression of the β2-adrenergic receptor;
(v) inhibiting synthesis or release of β2-adrenergic receptor, or agonists thereof from intracellular stores; or
(vi) increasing the rate of degradation of β2-adrenergic receptor, or agonists thereof.

It will be appreciated that each of mechanisms (i) to (vi) results in altering transmission at the receptor, and the activity thereof, to thereby negatively modulate the β2-adrenergic receptor. The negative modulator may be an antibody capable of altering receptor conformation/stability, or blocking the receptor's activity. The agent may be a β2-adrenergic receptor-selective partial agonist or a β2-adrenergic receptor-selective inverse agonist.

By the term “partial agonist”, we mean an agent, which binds with equal efficiency to both the inactive and active conformation of the β2-adrenergic receptor, maintaining the basal level of downstream effectors, such as cAMP.

By the term “inverse agonist”, we mean an agent, which stabilizes the inactive conformation of the β2-adrenergic receptor, and switches signalling pathways to lower the basal level of downstream effectors, such as cAMP.

However, preferably the agent is a β2-adrenergic receptor-selective antagonist.

By the term “antagonist”, we mean a molecule that blocks or prevents the ability of a given ligand (ie the catecholamines, adrenaline and noradrenaline) to bind to its receptor, ie the β2-adrenergic receptor.

Antagonists are characterized by their ability to inhibit the binding of another agent to the receptor. Suitably, the binding affinity value (Ki value) of the negative modulator for the β2-adrenergic receptor is less than about 100 nM, more suitably less than 80 nM, and more suitably less than 50 nM. Preferably, the Ki value of the negative modulator for the β2-adrenergic receptor is less than 30 nM, more preferably less than 15 nM, and more preferably less than 10 nM.

An example of a suitable antagonist which may be used in accordance with the invention is timolol. Timolol is 26 times more selective for β2-AR than for the β1-adrenergic receptor, and 759 times more selective for the β2-AR than for the β3-adrenergic receptor.

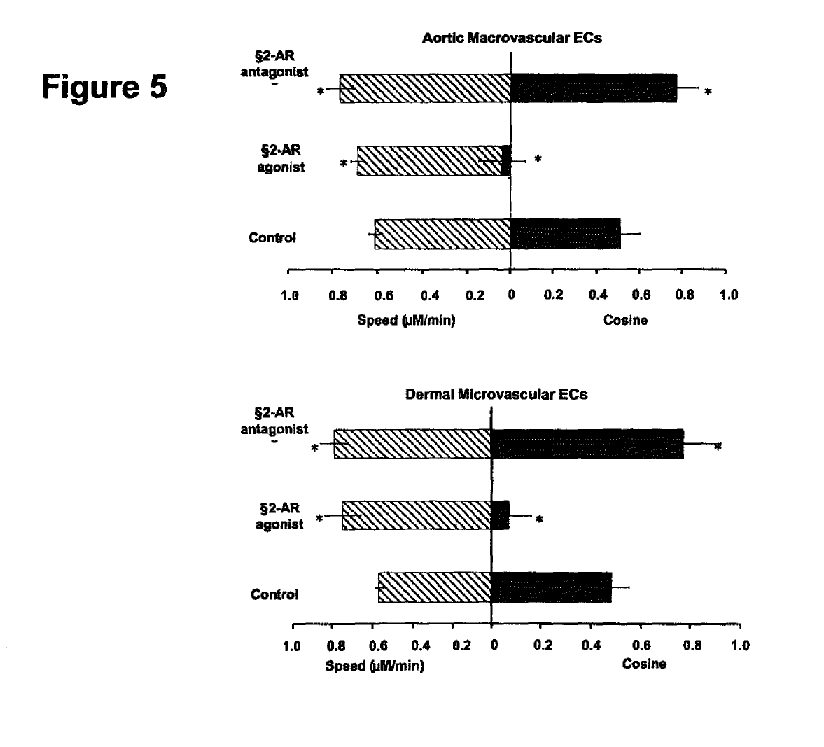

However, a preferred antagonist is ICI 118,551, which is shown in FIG. 7. The structure and preparation of ICI 118,551 is disclosed in EP 0,229,507. This compound has a Ki value for the β2-adrenergic receptor of about 1.2 nM, ie high selectivity. ICI 118,551 is highly selective for β2-adrenergic receptor at a concentration less than 100 nM, and will not bind to β1-adrenergic receptor unless much higher concentrations are used. ICI 118,551 is 550 times more selective for β2-AR than for the β1-adrenergic receptor, and 660 times more selective for β2-AR than for the β3-adrenergic receptor (Baker et al British J of Pharm., 144, 317 (2005). This paper states that the log K_{d} (dissociation constant) of ICI 118,551 for β1 is −6.52, that the log K_{d} of ICI 118,551 for β3 is −6.44, and that the log K_{d} of ICI 118,551 for β2 is −9.26. Hence, the log K_{d} is much lower for β2-adrenergic receptor than for the β1-AR or the β3-adrenergic receptor, and ICI 118,551 may therefore be described as being a β2-adrenergic receptor-selective antagonist.

It will be appreciated that the formation of new blood vessels arises primarily as a result of angiogenesis (ie a sprouting outgrowth from existing blood vessels) and in situ vasculogenesis (ie the differentiation of precursor cells into blood vessel networks). Furthermore, capillaries are responsible for passing blood and therefore nutrients around the body. Hence, medicaments according to the invention may be used to promote angiogenesis and organized, branched vasculature and/or treat conditions characterized by disorganized, unbranched vasculature.

Hence, by the term “organized vasculature”, we mean substantially branched blood vessels, or blood vessels with a normal or increased degree of branching, so as to promote blood supply to surrounding tissue.

By the term “disorganized vasculature”, we mean substantially unbranched blood vessels, or blood vessels with a reduced degree of branching, so as to impair blood supply to surrounding tissue.

Medicaments according to the invention may be used to increase capillary branching number (ie the average number of capillaries) and/or decrease capillary outgrowth (ie decrease their average length).

By the term “increased capillary branching number”, we mean the number of capillaries increases when in the presence of the medicament comprising an agent, which selectively modulates β2-adrenergic receptor conformation, or receptor activity, or activation thereof, when compared to the number of capillaries when the medicament is absent. The skilled technician will know how to measure whether capillary branching number has increased, as described in the Examples.

By the term “decreased capillary outgrowth”, we mean the average length of each capillary decreases when in the presence of the medicament comprising an agent, which selectively modulates β2-adrenergic receptor conformation, or receptor activity, or activation thereof, when compared to the length of capillaries when the medicament is absent. The skilled technician will know how to measure whether capillary outgrowth has decreased, as described in the Examples.

Angiogenesis involving increased capillary branching number and/or decreased capillary outgrowth results in a more organized, ordered and mature vasculature, as shown in FIGS. 4a and 4b. It should be appreciated therefore that a significant advantage of the invention is that the number of, and rate at which, capillaries are generated increase when exposed to the medicaments according to the invention, but that the vasculature retains an ordered or organized structure because the average length of each capillary is not increased.

In many clinical contexts, new blood vessel formation plays an important role in the supply of oxygen and nutrients to developing or damaged tissues. However, there are also many pathological conditions associated with new blood vessel formation. Examples of diseases associated with disorganized, unbranched blood vessel formation, which may be treated with medicaments according to the invention include cancer, where the development of disorganized and unbranched blood vessels is associated with tumour growth and propagation. Other examples of conditions that may be treated with medicaments of the invention include vasoproliferative retinopathies, such as proliferative diabetic retinopathy, retinopathy of prematurity (previously known as retrolental fibroplasia), and sickle cell retinopathy, ‘wet’ macular degeneration and other forms of choroidal neovascularisation, psoriasis, atopic dermatitis and many inflammatory conditions, such as rheumatoid- and osteo-arthritis and bronchitis.

The inventor believes that she has developed an understanding of the mechanisms by which β2-adrenergic receptor-selective agonists and β2-adrenergic receptor-selective antagonists control blood vessel formation and vasculature organisation. The promotion of an organized, branched vasculature is thought to be important in a number of clinical situations, for example bone fracture union and organ regeneration. Hence, preferably medicaments may be used in the treatment of organ regeneration, and bone fractures and breaks, and to assist bone fracture reunion.

Furthermore, the re-organisation of existing vasculature, as well as the inhibition of angiogenesis, is also thought to benefit rheumatoid arthritis sufferers, patients with diabetic retinopathy (vessel growth in the cornea can lead to blindness), bronchitis, as well as cancer, and in wound healing. Additionally, inflammatory, infectious and neoplastic disorders encompass the majority of dermatologic conditions, and are all characterized by excessive, disorganized and unbranched vasculature, and so may all be treated with medicaments according to the invention. Furthermore, many common skin disorders are also amenable to antiangiogenic therapy including psoriasis, atopic dermatitis/eczema, lupus and dermatomyositis, and so may also be treated with medicaments according to the invention. The inventor believes that agents, which selectively modulate the β2-adrenergic receptor may control some of the disorganized, unbranched vasculature seen in these diseases, and so patients would have a wide scope of possible clinical applications.

However, it is especially preferred that the medicament may be used to treat, ameliorate or prevent cancer or metastasis.

Although the inventor does not wish to be bound by any hypothesis, she believes that a notable difference between the effects of β2-adrenergic receptor-selective agonists and β2-adrenergic receptor-selective antagonists on vasculature development is the response of cells to an applied electric field. As shown in the Examples, endothelial cells and tumour cells are not influenced by an applied electric field (ie they are blinded to the electric field), thereby resulting in disorganized/arrested capillary structure, whereas the antagonist enhances their directional migration, and results in an organized vasculature. The inventor has appreciated that electric fields are found around tumours, and believes that by adding a β2-adrenergic receptor-selective antagonist to a tumour, it will be possible to modulate the electric potential across and around the tumour, to thereby promote ordered vascularisation, ie increase branching and organization. By promoting ordered vascularisation in and around a tumour, the inventor believes that it should be possible to improve the delivery of a drug to the tumour, resulting in higher tumour-drug concentrations and hence, higher systemic toxicity. Hence, treatment and prevention of cancer may be improved.

Hence, in a fifth aspect, there is provided an agent, which modulates an endogenous electric potential across a cell, for use in detecting, treating, ameliorating or preventing cancer.

In a sixth aspect, there is provided use of an agent, which modulates an endogenous electric potential across a cell, in the manufacture of a medicament for the treatment, amelioration or prevention of cancer.

In a seventh aspect, there is provided a method for detecting, treating, preventing, or ameliorating cancer in a subject, the method comprising administering, to a subject in need of such treatment, a therapeutically effective amount of an agent, which modulates an endogenous electric potential across a cell.

By the term “modulate an endogenous electric potential across a cell”, we mean alter the polarization state of the cell membrane, or hyperpolarize/depolarize the cell's membrane potential. The endogenous electric potential of the cell may be positively modulated (ie increased) or negatively modulated (ie decreased). The Examples describe how the endogenous electric potential across a cell may be detected, and one example is shown in FIG. 6.

Preferably, the cell, which has its endogenous electric potential modulated, is a cancer cell. The cell may be part of a tumour or a cancerous growth. The medicament may be used in the treatment of metastatic disease, ie the spread of cancerous cells around the body.

Preferably, the agent is adapted to selectively modulate β2-adrenergic receptor conformation, or receptor activity or activation thereof. The agent may be capable of increasing the endogenous electric potential across the cell. The agent may be a positive modulator of the β2-adrenergic receptor. For example, a positive modulator may be a β2-adrenergic receptor-selective agonist, for example, salbutamol.

The agent may be capable of decreasing the endogenous electric potential across the cell. The agent may be a negative modulator of the β2-adrenergic receptor. A negative modulator may be capable of enhancing the cell's ability to migrate directionally in the presence of an electric field. Examples of suitable negative modulators which may be used include any agent which lowers the basal levels of cellular cAMP, such as an inactive analog of cAMP, or rp-cAMP (ie a Protein kinase A inhibitor).

Preferably, the negative modulator is a β2-adrenergic receptor-selective antagonist. A suitable antagonist is ICI 118,551. The inventor has demonstrated that a β2-adrenergic receptor-selective antagonist can reduce the electric potential across not only in a cancer cell, but also across and around the vicinity of a tumour. In particular, the inventor was surprised to observe a decrease in electric potential across the associated vasculature in the immediate vicinity of a tumour, ie where blood enters the tumour. The inventor therefore believes that administration of the antagonist may be used in the detection or treatment of cancer.

It will be appreciated that agents, and medicaments according to the invention may be used in a monotherapy (ie use of an agent, which selectively modulates β2-adrenergic receptor conformation, or receptor activity or receptor activation alone), to promote organized, or branched vasculature and/or to treat conditions characterized by disorganized, or unbranched vasculature, such as cancer. Alternatively, agents and medicaments according to the invention may be used as an adjunct to, or in combination with, known therapies for promoting organized or branched vasculature and/or treating conditions characterized by disorganized or unbranched vasculature. For example, when the agent or medicament is used for the treatment of cancer, it may be used in combination with known chemotherapeutic agents, such as nocodazole, amscarine or cisplatin.

The agents and medicaments according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well tolerated by the subject to whom it is given, and preferably enables delivery of the agents across the blood-brain barrier.

Medicaments comprising agents according to the invention may be used in a number of ways. For instance, oral administration may be required, in which case the agents may be contained within a composition that may, for example, be ingested orally in the form of a tablet, capsule or liquid. Compositions comprising agents of the invention may be administered by inhalation (eg intranasally).

Compositions may also be formulated for topical use. For instance, creams or ointments may be applied to the skin, for example, directly to a tumour.

Agents according to the invention may also be incorporated within a slow or delayed release device. Such devices may, for example, be inserted on or under the skin, and the medicament may be released over weeks or even months. The device may be located at least adjacent the treatment site, eg a tumour. Such devices may be particularly advantageous when long-term treatment with agents used according to the invention is required and which would normally require frequent administration (eg at least daily injection).

In a preferred embodiment, medicaments according to the invention may be administered to a subject by injection into the blood stream or directly into a site requiring treatment. For example, the medicament may be injected into a cancer cell, or tumour mass in a cancer patient. Injections may be intravenous (bolus or infusion) or subcutaneous (bolus or infusion), or intradermal (bolus or infusion).

It will be appreciated that the amount of agent according to the invention that is required is determined by its biological activity and bioavailability, which in turn depends on the mode of administration, the physicochemical properties of the agent and whether the agent is being used as a monotherapy or in a combined therapy. The frequency of administration will also be influenced by the above-mentioned factors and particularly the half-life of the agent within the subject being treated.

Optimal dosages to be administered may be determined by those skilled in the art, and will vary with the particular agent in use, the strength of the preparation, the mode of administration, and the advancement of the disease condition, such whether the cancer has metastasized. Additional factors depending on the particular subject being treated will result in a need to adjust dosages, including subject age, weight, gender, diet, and time of administration.

Known procedures, such as those conventionally employed by the pharmaceutical industry (eg in vivo experimentation, clinical trials, etc.), may be used to establish specific formulations of the agents according to the invention and precise therapeutic regimes (such as daily doses of the agents and the frequency of administration).

Generally, a daily dose of between 0.001 μg/kg of body weight and 10 mg/kg of body weight of agent according to the invention may be used for promoting organized vasculature or treating conditions characterized by disorganized vasculature depending upon which agent is used. More preferably, the daily dose is between 0.01 μg/kg of body weight and 1 mg/kg of body weight, more preferably between 0.1 μμg/kg and 100μg/kg body weight, and most preferably between approximately 0.1 μg/kg and 10 μg/kg body weight.

Daily doses may be given as a single administration (eg a single daily injection). Alternatively, the agent may require administration twice or more times during a day. As an example, agents may be administered as two (or more depending upon the severity of the condition being treated) daily doses of between 0.07 μg and 700 mg (ie assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses.

Based on her findings that a β2-adrenergic receptor-selective agonist promotes angiogenesis (albeit disorganized, unbranched or immature vasculature) and decreases vessel branching, and that a β2-adrenergic receptor-selective antagonist promotes vessel branching, resulting in an organized/mature and branched vasculature, the inventor believes that the combined effect of these two agents may be harnessed and used in the manufacture of clinically useful compositions.

Hence, in an eighth aspect, there is provided a composition comprising therapeutically effective amounts of a β2-adrenergic receptor-selective positive modulator and a β2-adrenergic receptor-selective negative modulator, and optionally a pharmaceutically acceptable vehicle.

The invention also provides in a ninth aspect, a process for making the pharmaceutical composition according to the eighth aspect, the process comprising combining therapeutically effective amounts of a β2-adrenergic receptor-selective positive modulator and a β2-adrenergic receptor-selective negative modulator, and a pharmaceutically acceptable vehicle.

The inventor believes that she is the first to determine a medical use for the composition according to the eighth aspect.

Therefore, in a tenth aspect, there is provided a composition according to the eighth aspect, for use as a medicament.

In an eleventh aspect, there is provided a composition according to the eighth aspect, for use in promoting organized vasculature, or for treating, ameliorating or preventing conditions characterized by disorganized vasculature.

In a twelfth aspect, there is provided use of a composition according to the eighth aspect in the manufacture of a medicament for promoting organized vasculature, or for treating, ameliorating or preventing conditions characterized by disorganized vasculature.

In a thirteenth aspect, there is provided a method of promoting organized vasculature in a subject or for treating, preventing, or ameliorating a subject suffering from a condition characterised by disorganized vasculature, the method comprising administering, to a subject in need of said treatment, a composition according to the eighth aspect.

The β2-adrenergic receptor-selective positive modulator in the composition according to the eighth aspect may be a β2-adrenergic receptor-selective agonist. The β2-adrenergic receptor-selective agonist may be fenoterol, butoxamine, salbutamol, clenbuterol, formoterol, or salmeterol. However, a preferred β2-adrenergic receptor-selective agonist is salbutamol.

The β2-adrenergic receptor-selective negative modulator in the composition according to the eighth aspect may be a β2-adrenergic receptor-selective partial agonist or a β2-adrenergic receptor-selective inverse agonist. Preferably, the negative modulator is a β2-adrenergic receptor-selective antagonist, such as ICI 118,551.

While the inventor does not wish to be bound by any hypothesis, she believes that the β2-adrenergic receptor-selective agonist promotes angiogenesis (ie stimulates an increase in blood vessel length), but inhibits vessel branching leading to an immature or disorganized vasculature, and that the β2-adrenergic receptor-selective antagonist not only promotes angiogenesis and vessel branching, but also organizes the vasculature thus formed. This synergistic mechanism of the agonist and antagonist was surprising to the inventor, and could not have been predicted from the prior art, because until now, the effects of selective β2-adrenergic receptor agonists and antagonists on vasculature organisation had not been understood.

The positive and negative modulators may be administered contemporaneously (eg as a composition according to the eighth aspect of the invention). Hence, it will be appreciated that the composition according to the eighth aspect comprises both the positive modulator (eg an agonist) and the negative modulator (ie the antagonist), which are administered simultaneously to the subject being treated.

However, preferably the positive and negative modulators are administered to the subject sequentially. Hence, the negative modulator may be administered to the subject independently of the positive modulator.

If administered sequentially, the positive and negative modulators should be therapeutically active within the subject being treated at the same time. Whichever method is used, the subject is effectively being administered with both the agonist and antagonist, in order to take advantage of the dual effect of both agents in the body. Therefore, it is important that if the positive and negative modulators are administered independently, they are administered within a suitable time frame such that the subject contains therapeutically active concentrations of each modulator, or active metabolites thereof. Accordingly, the present invention extends to dual administration of the agonist and antagonist, either simultaneously or independently.

In one embodiment, the negative modulator may be administered to the subject followed by administration of the positive modulator. However, preferably the subject is treated with the positive modulator followed by the negative modulator independently. The negative modulator may be administered to the subject within 24 to 48 hours of the positive modulator. However, preferably the negative modulator is administered to the subject within 12 hours, more preferably within 6 hours, and even more preferably within 4 hours, of the positive modulator. It is envisaged that the negative modulator is administered to the subject within 1 to 2 hours of the positive modulator

The inventor believes that there may be some benefit in promoting rapid disorganized, unbranched angiogenesis in a subject by administering the agonist either independently, or followed by promoting ordered, branched angiogenesis by administration of the antagonist. She believes that this may improve a treatment regime by improving the vasculature in and around a treatment site, eg a tumour, and thereby increase drug uptake.

Furthermore, treating conditions characterised by disorganized or unbranched vasculature with the agonist and antagonist according to the invention is particularly useful because such therapy results in surprisingly synergistic actions. Furthermore, satisfactory therapy may be effected using lower doses than would be required in a monotherapy (ie use of the agonist or antagonist alone). This has the advantage that any toxic side-effects associated with high doses of the agonist and/or antagonist may be obviated or reduced. Using lower doses of the combined agents according to the invention are more efficient in treatment than would be required in a monotherapy, without compromising the efficacy of the treatment. The agonist and antagonists may be used to treat existing medical conditions (eg cancer), but may also be used when prophylactic treatment is considered medically necessary.

When a delayed release device is used to administer the composition of the eighth aspect, the device may be operable to release the positive and negative modulators contemporaneously, or sequentially. It is preferred that the delayed release device is adapted to release a therapeutically effective amount of the positive modulator (ie the agonist), followed by a therapeutically effective amount of the negative modulator (ie the antagonist).

A “therapeutically effective amount” of agent is any amount which, when administered to a subject, provides in a subject, promotion of organized vasculature, or prevents and/or treats disorganized vasculature.

For example, the therapeutically effective amount of agent used may be from about 0.07 μg to about 700 mg, and preferably from about 0.7 μto about 70 mg. It is preferred that the amount of agent is an amount from about 7 μg to about 7 mg, and most preferably from about 7 μg to about 700 μg.

A “subject” may be a vertebrate, mammal, or domestic animal, and is preferably a human being. Hence, medicaments according to the invention may be used to treat any mammal, for example human, livestock, pets, or may be used in other veterinary applications.

A “pharmaceutically acceptable vehicle” as referred to herein is any combination of known compounds known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, in a preferred embodiment, the pharmaceutical vehicle is a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, eg cellulose derivatives, preferably sodium carboxymethyl cellulose solution), alcohols (including monohydric alcohols and polyhydric alcohols, eg glycols) and their derivatives, and oils (eg fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intravenous and particularly subcutaneous injection. The agents according to the invention may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Given that the full sequence and crystal structure of the β2-adrenergic receptor has been determined, the inventor believes that it will be possible to selectively modulate the β2-adrenergic receptor using protein or peptide agents. A preferred means of using protein or peptide agents which selectively modulate the β2-adrenergic receptor for promoting organized or branched angiogenesis or for treating conditions characterized by disorganized or unbranched vasculature is to deliver a protein or peptide modulator to the site requiring treatment by means of gene therapy. For instance, gene therapy may be used to decrease expression of β2-adrenergic receptors, increase expression of enzyme(s) responsible for the degradation of endogenous β2-adrenergic receptor agonists, increase expression of a protein which promotes breakdown or de-sensitisation of β2-adrenergic receptors, increase expression of a protein which promotes breakdown of β2-adrenergic receptor agonists, or for the purposes of expressing a peptide inhibitor of β2-adrenergic receptor.

Therefore, according to a fourteenth aspect of the present invention, there is provided a delivery system for use in a gene therapy technique, said delivery system comprising a nucleic acid molecule encoding a protein which directly or indirectly selectively modulates β2-adrenergic receptor conformation, or receptor activity, or activation thereof, said nucleic acid molecule being capable of being transcribed to allow expression of said protein, and thereby promote organized vasculature or to treat conditions characterized by disorganized vasculature.

The delivery system according to the fourteenth aspect is suitable for achieving sustained levels of the protein which directly or indirectly selectively inhibits β2-adrenergic receptor activity over a longer period of time than would be possible for most conventional therapeutic regimes. The delivery system may be used to induce continuous protein expression from cells in a tissue requiring treatment, such as a tumour, or a wound, or a region of the skin suffering from psoriasis, that has been transformed with the nucleic acid molecule. Therefore, even if the protein has a short half-life as an agent in vivo, therapeutically effective amounts of the protein may be continuously expressed in the treated tissue. Furthermore, the delivery system of the invention may be used to provide the nucleic acid molecule (and thereby the protein which is an active therapeutic agent) without the need to use conventional pharmaceutical vehicles such as those required in tablets, capsules or liquids, as described herein. Preferably, the nucleic acid encoding the modulator protein is a DNA molecule.

The delivery system of the present invention is such that the nucleic acid molecule is capable of being expressed (when the delivery system is administered to a subject) to produce a protein that directly or indirectly has activity for selectively inhibiting β2-adrenergic receptor activity. By the term “directly”, we mean that the product of gene expression per se has the required activity (eg a protein with receptor-neutralising activity). By the term “indirectly”, we mean that the product of gene expression undergoes or mediates (eg as an enzyme) at least one further reaction to provide an agent effective for selectively inhibiting β2-adrenergic receptor activity, and thereby promoting organized vasculature.

The nucleic acid molecule may encode a protein which is a β2-adrenergic receptor-selective positive modulator, such as an agonist. However, preferably the nucleic acid molecule encodes a protein which is a β2-adrenergic receptor-selective negative modulator, such as an antagonist. The antagonist may be proteinaceous, and adapted to bind to, and negatively modulate, the receptor.

The nucleic acid molecule may be contained within a suitable vector to form a recombinant vector. The vector may for example be a plasmid, cosmid or phage. Such recombinant vectors are highly useful in the delivery systems of the invention for transforming cells with the nucleic acid molecule encoding the active agent or protein.

Recombinant vectors may also include other functional elements. For instance, recombinant vectors can be designed such that the vector will autonomously replicate in the cell. In this case, elements which induce DNA replication may be required in the recombinant vector. Alternatively, the recombinant vector may be designed such that the vector and recombinant DNA molecule integrates into the genome of a cell. In this case, DNA sequences which favour targeted integration (eg by homologous recombination) are preferred. Recombinant vectors may also have DNA coding for genes that may be used as selectable markers in the cloning process.

The recombinant vector may also further comprise a promoter or regulator to control expression of the gene, as required. The nucleic acid molecule may be (but is not necessarily) one that becomes incorporated in the DNA of cells of the subject being treated. Undifferentiated cells may be stably transformed leading to the production of genetically modified daughter cells (in which case regulation of expression in the subject may be required, eg with specific transcription factors or gene activators). Alternatively, the delivery system may be designed to favour unstable or transient transformation of differentiated cells in the subject being treated. When this is the case, regulation of expression may be less important because expression of the nucleic acid molecule will stop when the transformed cells die or stop expressing the protein.

The delivery system may provide the nucleic acid molecule to the subject without it being incorporated in a vector. For instance, the molecule may be incorporated within a liposome or virus particle. Alternatively, a purified DNA molecule (eg histone-free DNA) may be inserted into a subject's cells by a suitable means, eg direct endocytotic uptake. The nucleic acid molecule may be transferred to the cells of a subject to be treated by transfection, infection, microinjection, cell fusion, protoplast fusion or ballistic bombardment. For example, transfer may be by ballistic transfection with coated gold particles, liposomes containing the nucleic molecule, viral vectors (eg adenovirus) and means of providing direct DNA uptake (eg endocytosis) by application of the DNA molecule directly to the tumour, topically or by injection.

The invention also provides first and second medical uses of the delivery system according to the invention.

Hence, in a further aspect, there is provided a delivery system according to the fourteenth aspect, for use as a medicament.

The invention provides in a still further aspect, the delivery system according to the fourteenth aspect for use for the promotion of organized vasculature and/or for the treatment, amelioration or prevention of a condition characterized by disorganized vasculature.

In a still further aspect, the invention provides use of the delivery system according to the fourteenth aspect in the manufacture of a medicament for the promotion of organized vasculature and/or for the treatment, amelioration or prevention of a condition characterized by disorganized vasculature.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

FIG. 1 is a bar chart showing the effect of β2-adrenergic receptor (AR) agonists and antagonists on endothelial cell migration by monitoring the motility of single endothelial cells over one hour;

FIG. 2A is a graph showing the results of a wound healing assay that was performed in order to demonstrate the effect of β2-AR agonists or antagonists on the migration of endothelial cells from the edge of a wound created in aortic macrovascular cells;

FIG. 2B is a graph showing the results of a wound healing assay that was performed in order to demonstrate the effect of β2-AR agonists or antagonists on the migration of endothelial cells from the edge of a wound created in dermal microvascular endothelial cells;

FIG. 3 shows photographs at 10× magnification of the effect of adding β2-AR agonists and antagonists on endothelial cell formation plated on top of Matrigel, which were observed microscopically every 4 hours until 21 hours;

FIG. 4a shows photographs showing the effects of β2-AR agonists and antagonists on vessel formation in three dimensions. An organotypic culture was established and capillary outgrowth was observed from the cut edges of aortic rings;

FIG. 4b shows images of the effects of β2-AR agonists and antagonists on angiogenesis in a chick chorioallantoic membrane (CAM) in vivo assay. The effects of both β2-AR agonists and antagonists on angiogenesis and vessel branching can be seen.

FIG. 5 shows the results of an experiment to determine if human microvascular and aortic macrovascular endothelial cells had the capacity to sense and respond to an applied physiological electric field of 100 mV/mm. Cells were plated in specialised galvanotaxis chambers, and cell migration was monitored and analysed as described;

FIG. 6 shows the effects of β2-AR agonists and antagonists on the directional migration of a mammary adenocarcinoma cell line, MTLn3. Cells were plated in the presence and absence of β2-AR ligands and electric field-mediated directional migration was determined;

FIG. 7 shows the chemical structure of a selective β2-AR antagonist, ICI 118,551;

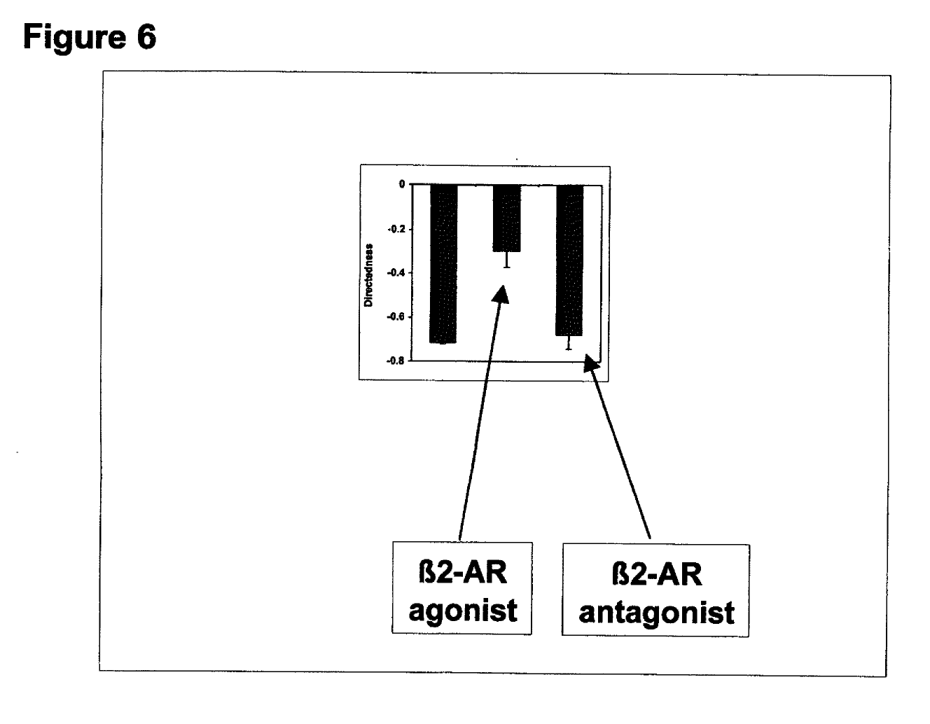

FIG. 8 is a barchart showing the in vivo effects of a β2-AR agonist and a β2-AR antagonist on tumour size in mice three days and seven days after administration;

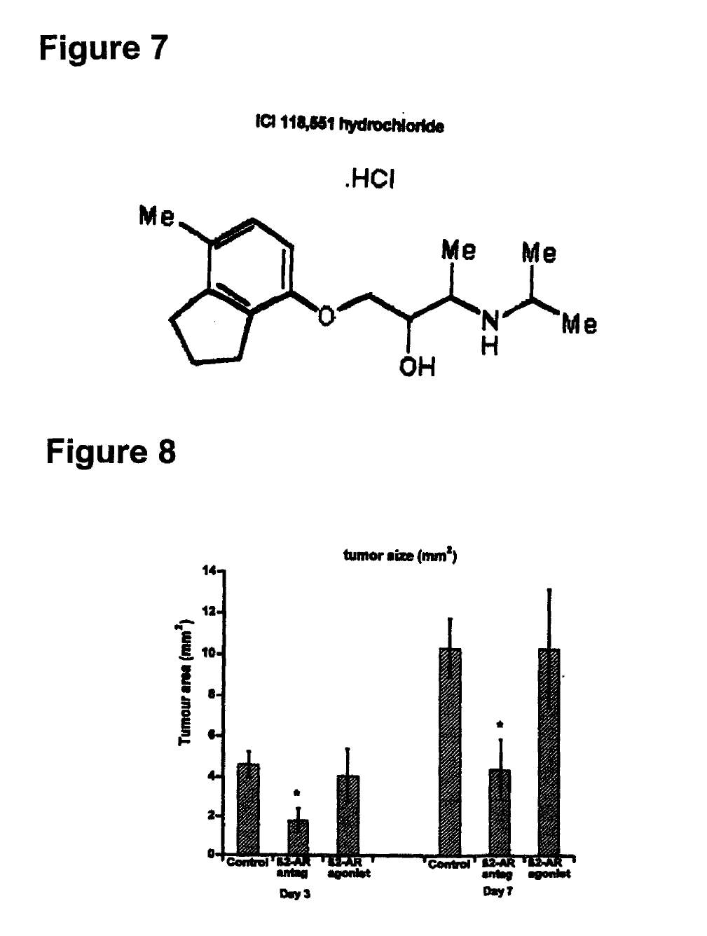

FIG. 9 is a barchart showing the weight of tumours eleven days following administration of either the β2-AR agonist or β2-AR antagonist with respect to the control; and

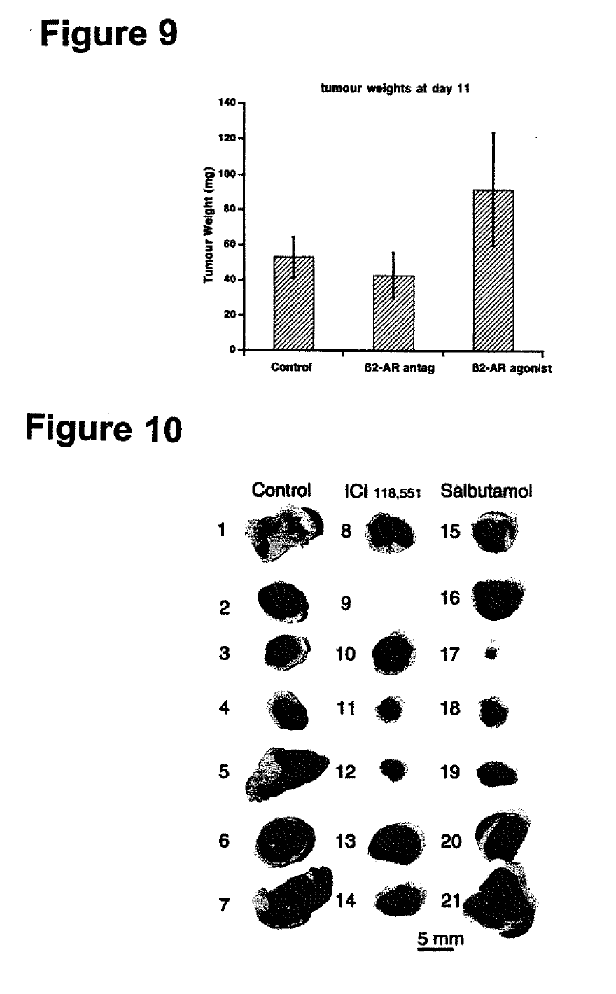

FIG. 10 is a photograph of the tumours at eleven days referred to in FIG. 9.

The Figure shows the size of untreated tumours (control), tumours treated with antagonist (ICI 118,551) and tumours treated with agonist (salbutamol). The size of the tumours is indicated by the 5 mm scale at the bottom of the Figure.

### EXAMPLES

### Materials

The following experiments included the use of a selective β2-adrenergic receptor antagonist, ICI 118,551 (Tocris Bioscience, UK), the structure of which is shown in FIG. 7. This compound is a highly selective β2-adrenergic receptor antagonist, having Ki values of 1.2, 120, and 257 nM for β2-, β1-, and β3-adrenergic receptors, respectively (Bilski et al (1983) J. Cardiovasc. Pharmacol 5, 430). The experiments also used a selective β2-adrenergic receptor agonist, salbutamol (Sigma-Aldrich, UK).

Further materials included Dermal microvascular endothelial cells (Cascade Biologics, UK); Aortic macrovascular endothelial cells (Cascade Biologics, UK); Medium 131 containing microvascular growth supplement (MVGS) (Cascade Biologics, UK); Medium 200 containing low serum growth supplement (LSGS) (Cascade Biologics, UK); and Matrigel (BD Biosciences, Belgium).

### Methods

### Endothelial Cell Growth

Dermal microvascular and aortic macrovascular endothelial cells were maintained in a 37° C. incubator with 5% CO₂ in Medium 131 containing MVGS or medium 200 containing LSGS, respectively. Passage 3-7 cells were used for all experiments.

### Single Cell Migration Assay

Glass bottomed 35 mm dishes (MatTek Corporation, Ashland, Mass.), were coated with collagen I (60 μg/ml) (Cohesion Technologies, Palo Alto, Calif.) in PBS for 1 hour at 37° C. Endothelial cells were plated at a density of 50 cells/mm² for 2 hours at 37° C., and then incubated with medium alone (control) or with medium containing either 10 nM β2-AR antagonist (ICI 118,551, Tocris Bioscience) or 1 μM β2-AR agonist (salbutamol). The 35 mm glass-bottomed dishes were placed in a heating chamber, designed to maintain the media between 35° C. to 37° C., secured to the stage of an inverted Nikon Diaphot microscope. Individual cell migration was monitored over a 1 hour period at 37° C., as described previously in Pullar and Isseroff (2005) J Cell Sci 118 (Pt 9), 2023-2034. Time-lapse images of the cell migratory response were digitally captured every 10 minutes by Q-Imaging Retiga-EX cameras (Burnaby, BC, Canada) controlled by a custom automation written in Improvision Open Lab software (Lexington, Mass.) on a Macintosh G4. After each cell's center of mass was tracked using the Open Lab software, migration speed and distance were calculated and imported to Excel (Microsoft Corporation, Redmond, Wash.). Significance was taken as P<0.01, using Student's t test (unpaired) to compare the means of two cell populations.

### Scratch Assay

Endothelial cells were grown to confluence in medium on plastic tissue culture dishes (Fisher Scientific, Pittsburgh, Pa.) coated with 60 μg/ml collagen I (Vitrogen 100, Collagen Corp., Palo Alto, Calif.) in PBS for 1 hour at 37° C. A sterile pipette tip was used to scratch a 1 mm-wide wound along the center of the dish and the medium was replaced with either medium alone or medium containing either 10 nM β2-AR antagonist (ICI 118551) or 1 μM β2-AR agonist (salbutamol). Demarcated areas of each wound were photographed on an inverted Nikon Diaphot microscope at the time of wounding (time 0) up to wound healing as described in Pullar et al., (2003) J Biol Chem 278 (25), 22555-22562. Image J was used to measure each demarcated wound area at time 0 and 20 hours post wounding and the results were averaged to calculate the % healing for control and β2-AR ligand-treated cells.

### In Vitro Model of Angiogenesis

An experimental in vitro model was employed to observe the effect of β2-AR ligands (ie agonists and antagonists) on endothelial cell vessel formation. Endothelial cells were plated on Matrigel (as described in Kubota et al. (1988) J Cell Biol 107 (4), 1589-1598) in media alone or media containing either 10 nM β2-AR antagonist (ICI 118551) or 1 μM β2-AR agonist (salbutamol). The endothelial cells plated on the top of the Matrigel were maintained in a 37° C. incubator with 5% CO₂ Tubule formation was observed every 4 hours up to 21 hours using an inverted Nikon Diaphot microscope.

### Rat Aortic Ring Assay

A rat aortic ring assay (as described in Nicosia and Ottinetti (1990) Lab Invest 63 (1), 115-122; and Diglio et al (1989) Lab Invest 60 (4), 523-531) was used to study the effect of β2-AR ligands on vessel formation. Rat aorta was obtained from animals that were euthanised under a Home Office Approved Schedule 1 method. The aorta was cut into small rings with sterile scissors and each piece was submerged under a 50 μl bead of Matrigel in the bottom of each in a 24-well dish. Twenty minutes later 1 ml of medium alone (control) or medium containing either 10 nM β2-AR antagonist (ICI 118,551) or 1 μM β2-AR agonist (salbutamol) was added to each well. Vessel growth was observed using an inverted Nikon Diaphot microscope every 30 hours and images were captured as described in Pullar et al (2005) J Cell Sci 118 (Pt 9), 2023-2034; Pullar et al (2006) Mol Biol Cell and Pullar et al (2001) Cell Motil Cytoskeleton 50 (4), 207-217.

### Chick Chorioallantoic Membrane (CAM) Assay

Fertilised chicken eggs were obtained within the first two days of embryonic development, and were cleaned and incubated at 37° C. for 24 hours. On day 3 of development, 1-2 ml of albumin was removed from the apex end of each egg with a sterile needle. A window of 1.5×1.5 cm was created in the shell with a Dremmel tool, and was sealed with clear adhesive tape. The eggs were incubated for a further 5 days, at which time water alone, or containing 10 μM salbutamol, 10 μM forskolin, or 10 nM ICI 118,551, was evaporated onto coverslips and placed on the exposed CAM. Images of the exposed vasculature were captured 48 hours later using a Nikon microscope/imaging system. Scale bar=1 mm. Bifurcation points were counted in four fields for the control/treatment groups.

### Galvanotaxis

Endothelial cells were seeded at low density in medium within electrostatic chambers for 2-3 hours prior to exposure to an electric field (EF). A roof consisting of a No 1 cover slip was applied and sealed on top of the chamber, as previously described (in Pullar et al (2005) J Cell Sci 118 (Pt 9), 2023-2034; Pullar et al (2006) Mol Biol Cell and Pullar et al (2001) Cell Motil Cytoskeleton 50 (4), 207-217). A direct current EF of 100 mV/mm was applied through agar bridges connecting silver/silver chloride electrodes to the culture medium at either side of the chamber. The chamber was placed on an inverted microscope with temperature controlled at 37° C. The experiments were performed in media alone or media containing either 10 nM β2-AR antagonist (ICI 118,551) or 1 μM β2-AR agonist (salbutamol).

### Time-Lapse Video Microscopy and Quantification of Cell Migration

Time-lapse images were recorded every 10 minutes over 1 hour and analyzed with Improvision software as previously described (Pullar et al (2005) J Cell Sci 118 (Pt 9), 2023-2034; Pullar et al (2006) Mol Biol Cell and Pullar et al (2001) Cell Motil Cytoskeleton 50 (4), 207-217). Migration directedness (cosine Q) shows how a cell directionally migrated within the field, where θ is the angle between the EF vector and a straight line connecting the start and end position of a cell. A cell moving perfectly towards the cathode would have a directedness of 1, and a cell moving perfectly along the field lines toward the anode would have a directedness of −1. Therefore, the average of directedness values of a population of cells gives an objective quantification of how, directionally, cells have moved within the electric field. A group of cells migrating randomly would have an average directedness value of 0. Migration rate was analyzed with the following two parameters, ie trajectory speed (μm/min) is the total length of the migration trajectory of a cell divided by the given period of time (ie 60 minutes).

### Results

### β2-AR Agonists and Antagonists Promote Endothelial Single Cell Migration

Endothelial cell sprouting from existing vasculature is an essential process in angiogenesis and requires endothelial cell migration. To determine the effect of β2-AR agonists and antagonists on endothelial cell migration, the motility of single endothelial cells was monitored over one hour.

Referring to FIG. 1, there is shown the effect of β2-AR agonists and antagonists on endothelial cell migration. As can be seen in FIG. 1, the addition of either a β2-AR agonist or antagonist increased endothelial cell motility by 20-30% in both dermal microvascular and aortic macrovascular endothelial cells. Hence, administration of either form of β2-adrenergic receptor selective modulator (ie the agonist or the antagonist) results in the promotion of angiogenesis.

### β2-AR Agonists and Antagonists Promote the Healing of a Wound in a Confluent Sheet of Endothelial Cells

To investigate the effect of β2-AR agonists or antagonists on the migration of endothelial cells from the edge of a wound created in a confluent sheet of cells, a wound healing assay was performed.

Referring to FIGS. 2A and 2B, there are shown the results of wound healing assays that were performed to demonstrate the effect of β2-AR agonists or antagonists on the migration of endothelial cells from the edge of a wound created in aortic macrovascular cells and in dermal microvascular endothelial cells, respectively. As shown in FIG. 2A, within 6 hours of wounding, β2-AR agonists and antagonists both accelerated wound closure by 20-30% in aortic macrovascular cells. As shown in FIG. 2B, in dermal microvascular endothelial cells, the rate of wound closure was increased by 40% in the presence of either a β2-AR agonist or antagonist. Hence, administration of either form of β2-adrenergic receptor-selective modulator (ie the agonist or the antagonist) results in an increase in the rate of wound closure.

### β2-AR Antagonists Promoted an Organized Vascular Network, While β2-AR Agonist Delayed Vessel Formation, Resulting in a Dis-Organized, Immature Vasculature

Angiogenesis requires the migration, proliferation, elongation and re-orientation of endothelial cells to promote tubule formation. While both β2-AR-selective agonists and antagonists appear to promote endothelial cell formation, their effects on tubule formation are unknown. Therefore, endothelial cells were plated on top of Matrigel in the absence or presence of either a β2-AR agonist or antagonist and observed microscopically every 4 hours until 21 hours.

Referring to FIG. 3, there are shown photographs at 10× magnification of the effect of adding β2-AR agonists and antagonists on endothelial cell formation plated on top of Matrigel at 12 hours and at 21 hours. As can be seen in FIG. 3, untreated endothelial cells organized into a capillary-like network within 21 hours (top right hand photo). However, in the presence of the β2-AR antagonist, this process was significantly accelerated and a highly organized, mature capillary network was observed within only 12 hours (bottom left photo). In contrast, a β2-AR agonist delayed the process, and by 21 hours, the endothelial cells had created a disorganized, immature capillary network (bottom right photo).

Hence, the inventor has demonstrated that β2-AR-selective antagonists promote a modest, but very organized capillary outgrowth, while β2-AR agonists accelerated capillary outgrowth creating a larger, immature vasculature. Therefore, surprisingly, the inventor believes that she is the first to notice that that the addition of β2-AR antagonists promotes an organized, structured vascular network, whereas β2-AR agonist delayed vessel formation, resulting in a dis-organized, immature vasculature. The inventor believes that this is a significant and important observation.

### β2-AR Antagonists Promoted a Modest, Organized Capillary Outgrowth, While β2-AR Agonists Accelerated Capillary Outgrowth Creating a Larger, Immature Vasculature

In order to observe the effects of β2-AR agonists and antagonists on capillary vessel formation in three dimensions (ie a more similar situation to angiogenesis in vivo), an organotypic culture was established, and capillary outgrowth was observed from the cut edges of aortic rings.

Referring to FIG. 4a, there are shown photographs showing the effects of β2-AR agonists and antagonists on vessel formation in three dimensions. As shown in FIG. 4a, capillary outgrowth was observed from the cut edges of untreated aorta after 90 hours. Addition of a β2-AR-selective agonist significantly increased the length and number of the capillaries growing from the cut aorta in an irregular and dis-organized fashion. However, in contrast, FIG. 4a shows that the addition of a β2-AR-selective antagonist decreased the length of the capillaries growing from the cut edge. Furthermore, the capillaries growing from the aorta in the presence of β2-AR antagonist appear to be more organized and restricted in growth from the cut edge of the tissue. Hence, the inventor has shown that the use of a selective β2-AR antagonist causes faster growth of capillaries than the control, and that these capillaries are shorter and more organized in structure than upon administration of a β2-AR agonist. This was totally unexpected.

As demonstrated in the data, a β2-AR antagonist enhances angiogenesis and vascular branching, while a β2-AR agonist or an agent that increases intracellular cAMP, arrests vascular development in the CAM assay. In order to observe the effect of β2-AR agonists and antagonists on vascular development in vivo, the chick chorioallantoic membrane assay was used as shown in FIG. 4b. The addition of either 10 μM β2-AR agonist or 10 μM forskolin, which activates adenylyl cyclase increasing intracellular cAMP, to day 8 chick embryos decreased vessel branching and arrested vascular development. The number of vessel bifurcation points decreased from 15 (in the control) to 4 (β2-AR agonist-administered embryos) and 5 (forskolin-administered embryos), respectively. In contrast, the addition of a β2-AR antagonist to day 8 chick embryos significantly enhanced vascular density and branching (vessel bifurcation points) to >50, as shown in FIG. 4b.

Hence, the inventor has demonstrated that the addition of a β2-AR antagonist to an already established vasculature can increase angiogenesis and vessel branching, resulting in a highly organized vasculature. In contrast, the addition of a β2-AR agonist or an agent that increases intracellular cAMP (ie forskolin) can destabilise an already established vasculature, resulting in a decrease in vessel branching producing an immature vasculature and an arrest of blood vessel growth.

A β2-AR Antagonist Enhances the Ability of Endothelial Cells to Sense and Respond to an Applied Electric Field, While a β2-AR Agonist Appears to “Blind” the Cells to this Guidance Cue

Electric potentials are present in all developing and regenerating animal tissues, and are essential to regulate the appropriate cell behaviours during embryogenesis, wound healing and tissue regeneration. Highly proliferating cells and cancerous cells have altered electric potentials that could contribute to the loss of normality, and hence, metastasis. Numerous cells have the ability to sense and respond to applied electric fields in vitro by migrating directionally, including endothelial cells.

In order to determine if human microvascular and aortic macrovascular endothelial cells had the capacity to sense and respond to an applied physiological electric field of 100 mv/mm, cells were plated in specialised galvanotaxis chambers, and migration was monitored and analysed.

Referring to FIG. 5, there is shown the results of these experiments. As shown in FIG. 5, while both dermal microvascular and aortic macrovascular endothelial cells have the capacity to sense and respond to an applied electric field by migrating towards the cathode (cosine=0.5), the presence of β2-AR agonists and antagonists significantly alter this process compared to the control. The addition of a β2-AR-selective antagonist enhances the ability of endothelial cells to sense and respond to the applied electric field and increases directional migration by as much as 60% in both aortic and dermal cells. In contrast, the application of a β2-AR agonists appears to render the endothelial cells so that they are not influenced by the applied electric field and the cells migrate randomly (cosine=0).

A β2-AR Antagonist Enhances the Ability of Tumour Cells to Sense and Respond to an Applied Electric Field, While a β2-AR Agonist Appears to “Blind” the Cells to this Guidance Cue

Cancer cell lines can also sense and respond to an applied electric field by migrating towards the anode or the cathode of an applied electric field. Additionally, there is some evidence that metastatic cells have an altered ability to sense and respond to an applied electric field. While the inventor does not wish to be bound by any hypothesis, she believes that the disregard of this essential guidance cue could correlate with metastasis. Therefore, in order to determine the effect of β2-AR agonists and antagonists on the directional migration of a mammary adenocarcinoma cell line, MTLn3 cells (Rohde-Schulz and Lichtner (1995) Invasion Metastasis 15 (1-2), 1-10), were plated in the presence and absence of β2-AR ligands, and electric field mediated directional migration was determined as described previously (Pullar et al., (2005) J Cell Sci 118 (Pt 9), 2023-2034; Pullar et al., (2006) Mol Biol Cell; Pullar et al., (2001) Cell Motil Cytoskeleton 50 (4), 207-21.

Referring to FIG. 6, there is shown the effects of β2-AR-selective agonists and antagonists on the directional migration of a mammary adenocarcinoma cell line, MTLn3. As shown in the Figure, in contrast to endothelial cells, MTLn3 cells migrate towards the anode of the applied electric field. While the application of a β2-AR antagonist to the tumour cells appears to have little effect on galvanotaxis, the β2-AR agonist appears to mimic a “disregard” of electric field guidance, with directional migration being reduced by more than 50%.

### DISCUSSION AND CONCLUSIONS

The inventor has clearly demonstrated that β2-AR-selective antagonists promote the formation of an organized, branched, and mature vasculature and enhance the ability of both endothelial and tumour cells to sense and respond to homing/guidance cues, characteristic of “normal” cell behaviour. The inventor believes therefore that β2-AR-specific antagonists could be a potential future treatment for any disease characterised by disorganized vasculature and metastasis. The promotion of an organized and branched vasculature would enhance drug delivery and increase the susceptibility of the tumour to chemotherapy. On the other hand, β2-AR-selective agonists appear to promote a more disorganized, unbranched, immature vasculature of tumours. However, the agonist does stimulate an increase in blood vessel length , which in some treatment regimes may be beneficial, for example, when an agonist is administered in combination with an antagonist.

### β2-AR/Melanoma In-Vivo Study

Twenty one, 5 week-old female SKH1 mice (immunocompetent, hairless albino strain, Charles River, Wilmington, Mass.) weighing 18 g-25 g were divided into 3 groups of 7 mice. They were implanted, sub-cutaneously, on the left flank, with alzet pumps 1002 (Cupertino, Calif.) containing either Hank's buffered salt solution (HBSS) (Gibco, Grand Island, N.Y.) containing 0.2% ascorbic acid (sigma, St Loius, Mo.) alone (ie the control group), or either (i) a β2-AR-selective antagonist, ICI 118,551 (Tocris, Ellisville, Mo.) delivered at a rate of 0.75 mg/kg/day (antagonist group) or (ii) a β2-AR-selective agonist, salbutamol (Sigma) delivered at a rate of 3 mg/kg/day (agonist group).

Four days after administration, all mice were injected sub-dermally, on the right thigh (Nuccitelli et al, BBRC 343:351-360, 2006), with 10 μl of sterile HBSS containing 0.5×10⁶ B16-BL6 melanoma cells, derived from a highly metastatic melanoma cell line (Qian et al, Mol Med 13 (3-4): 151-159, 2007, Poste et al, Cancer Research, 40: 1636-1644, 1980). For all studies, the mice were examined, photographed, and weighed under inhalation anesthesia using 1.4% isoflurane in oxygen. All procedures were approved by the local IACUC.

Melanomas were imaged daily by both transillumination and surface photography at 2× magnification, using an upright Nikon microscope controlled by Pixera software (San Jose, Calif.). Mice were sacrificed after 11 days by CO₂ asphyxiation, and the tumor, lungs and the liver were excised and fixed in 10% buffered formalin for 7 days. The surface area of the tumour on each mouse was calculated from the calibrated images of the tumours after 3 and 7 days post-injection as shown in FIG. 8.

All of the control mice developed tumours that were clearly visible after 3 days. However, while the control group exhibited tumours with a surface area of 4.6+/−1-0.63 mm², β2-AR antagonist-treated mice exhibited significantly smaller tumours (P<0.001), with a surface area of only 1.8+/−0.61 mm², as shown in FIG. 8. In addition, two of the β2-AR antagonist-treated mice failed to show any signs of tumour development after 3 days, which was most surprising. In contrast, all of the β2-AR agonist-treated mice developed tumours after 3 days with an average surface area of 4.1+/−1.3 mm².

With reference to FIG. 8, as expected, all tumours were larger after 7 days compared their size after 3 days. The control group exhibited tumours with an average surface area of 10.4+/−1.4 mm² after 7 days. The tumours on the β2-AR antagonist-treated mice were significantly smaller with an average surface area of only 4.4+/−1.5 mm² than those tumours exhibited by control mice after 7 days, ie the tumour size caused by the melanoma cells injected into control mice after only 3 days. In addition, most surprisingly one mouse treated with β2-AR antagonist displayed no visible signs of any tumour development after 7 days, or even by the end of the study (ie 11 days). In contrast, the tumours exhibited by β2-AR agonist-treated mice were not significantly different from the tumours on the control mice, with an average surface area of 10.4+/−2.9 mm². However, it is noteworthy that one of the β2-AR agonist-treated mice had developed a very large tumor, with a surface area of 24.8 mm².

All mice were sacrificed by CO₂ asphyxiation, 11 days post melanoma cell injection. The tumours were excised from their sub-dermal location, fixed in saline-buffered 10% formalin for 10 days, and then weighed. The average weight of the tumours from each group was calculated and statistically analysed. The data is shown in FIG. 9. As can be seen in FIG. 9, the average weight of tumours treated with the β2-AR-selective antagonist was statistically lower than that for the control group.

Finally, FIG. 10 illustrates the tumours themselves at eleven days. Clearly, the tumours treated with the antagonist are much smaller than those in the control sample.

### Conclusions

The data for the in vivo study strongly supports the hypothesis that β2-AR-selective antagonists are able to slow down tumour growth, reducing both tumour size and weight. Advantageously, unlike in previous studies, with the present invention, the application of a β2-adrenergic receptor antagonist reduced the normal growth of the tumour, rather than just decreasing the increase in tumour growth that was produced by the application of an external stress, ie no external stress had to be applied before administration of the antagonist in order to achieve a therapeutic effect.

1.-39. (canceled)

40. A method for promoting organized vasculature, or for treating, ameliorating or preventing a condition characterized by disorganized vasculature in a subject, the method comprising administering, to a subject in need of such treatment, a therapeutically effective amount of an agent, which selectively modulates β2-adrenergic receptor conformation, or receptor activity, or activation thereof.

41. A method according to claim 40, wherein the agent is a positive modulator.

42. A method according to claim 41, wherein the binding affinity value (Ki value) of the positive modulator for the β2-adrenergic receptor is less than about 100 nM.

43. A method according to claim 41, wherein the positive modulator is a β2-adrenergic receptor-selective agonist.

44. A method according to claim 43, wherein the β2-adrenergic receptor-selective agonist is fenoterol, butoxamine, salbutamol, clenbuterol, formoterol, or salmeterol.

45. A method according to claim 43, wherein the β2-adrenergic receptor-selective agonist is salbutamol.

46. A method according to claim 40, wherein the agent is a negative modulator.

47. A method according to claim 46, wherein the binding affinity value (Ki value) of the negative modulator for the β2-adrenergic receptor is less than about 100 nM.

48. A method according to claim 46, wherein the agent is a β2-adrenergic receptor-selective partial agonist or a β2-adrenergic receptor-selective inverse agonist.

49. A method according to claim 46, wherein the agent is β2-adrenergic receptor-selective antagonist.

50. A method according to claim 49, wherein the antagonist is ICI 118,551 or timolol.

51. A method according to claim 40, wherein the promotion of organized vasculature comprises increasing capillary branching number (i.e., the average number of capillaries) and/or decreasing capillary outgrowth (i.e., decrease their average length).

52. A method according to claim 40, which is for treating, ameliorating or preventing cancer, vasoproliferative retinopathies, such as proliferative diabetic retinopathy, retinopathy of prematurity, sickle cell retinopathy, ‘wet’ macular degeneration or other forms of choroidal neovascularisation, psoriasis, atopic dermatitis/eczema, lupus, dermatomyositis, inflammatory conditions, rheumatoid- or osteo-arthritis, bronchitis, organ regeneration, bone fractures or breaks, or to assist bone fracture reunion.

53. A method according to claim 40, which is for treating, ameliorating or preventing cancer or metastasis.

54. A composition comprising therapeutically effective amounts of a β2-adrenergic receptor-selective positive modulator and a β2-adrenergic receptor-selective negative modulator, and optionally a pharmaceutically acceptable vehicle.

55. A composition according to claim 54, wherein the β2-adrenergic receptor-selective positive modulator is a β2-adrenergic receptor-selective agonist, and the β2-adrenergic receptor-selective negative modulator is a β2-adrenergic receptor-selective antagonist.

56. A method of promoting organized vasculature in a subject or for treating, preventing, or ameliorating a subject suffering from a condition characterised by disorganized vasculature, the method comprising administering, to a subject in need of said treatment, therapeutically effective amounts of a β2-adrenergic receptor-selective positive modulator and a β2-adrenergic receptor-selective negative modulator.

57. A method according to claim 56, wherein the positive and negative modulators are administered to a subject simultaneously.

58. A method according to claim 56, wherein the positive and negative modulators are administered to a subject sequentially.

59. A method according to claim 58, wherein the positive modulator is administered to the subject followed by administration of the negative modulator.

60. A delivery system for use in a gene therapy technique, said delivery system comprising a nucleic acid molecule encoding a protein which directly or indirectly selectively modulates β2-adrenergic receptor conformation, or receptor activity, or activation thereof, said nucleic acid molecule being capable of being transcribed to allow expression of said protein, and thereby promote organized vasculature or to treat conditions characterized by disorganized vasculature.

61. A delivery system according to claim 60, wherein the nucleic acid molecule encodes a protein which is a β2-adrenergic receptor-selective positive modulator, such as an agonist.

62. A delivery system according to claim 60, wherein the nucleic acid molecule encodes a protein which is a β2-adrenergic receptor-selective negative modulator, such as an antagonist.

63. A method for detecting, treating, preventing, or ameliorating cancer in a subject, the method comprising administering, to a subject in need of such treatment, a therapeutically effective amount of an agent, which modulates an endogenous electric potential across a cell.

64. A method according to claim 63, wherein the cell, which has its endogenous electric potential modulated, is a cancer cell.

65. A method according to claim 63, wherein the cell is part of a tumour or a cancerous growth.

66. A method according to claim 63, wherein the agent is adapted to selectively modulate β2-adrenergic receptor conformation, or receptor activity, or activation thereof.
